(19) United States
(12) Patent Application Publication
(10) Pub. No.: US 2018/0049971 A1
(43) Pub. Date: Feb. 22, 2018

(54) Multi-Purpose Moisturizer Composition

(71) Applicant: Whitney Druilhet, Blencoe, IA (US)

(72) Inventor: Whitney Druilhet, Blencoe, IA (US)

(21) Appl. No.: 15/680,462

(22) Filed: Aug. 18, 2017

Related U.S. Application Data
(60) Provisional application No. 62/377,036, filed on Aug. 19, 2016.

(51) Int. Cl. A61K 8/92; A61Q 19/00; A61K 8/98; A61K 8/67; A61K 8/06; A61Q 5/12

(52) U.S. Cl. CPC A61K 8/922; A61Q 19/007; A61K 8/985; A61Q 5/12; A61K 8/925; A61K 8/06; A61K 8/678

(57) ABSTRACT

A multi-purpose moisturizer composition. In one embodiment, the multi-purpose moisturizer has the form of a cream and is composed of a plurality of butter compounds such as, for example, rhea butter, cocoa butter, and jojoba butter, combined with a plurality of essential oils such as, for example, sweet almond oil, castor oil, argan oil, coconut oil, grape seed oil, and the like. In another embodiment, the multi-purpose moisturizer has the form of an oil and is composed of a plurality of essential oils such as, for example, hemp seed oil, vitamin E oil, chamomile oil, and the like. The cream or the oil embodiments of the multi-purpose moisturizer may be applied to an area of dry skin in order to alleviate itching and irritation while returning the skin to a healthy moisture level.

### CROSS REFERENCE TO RELATED APPLICATIONS

This application claims the benefit of U.S. Provisional Application No. 62/377,036 filed on Aug. 19, 2016. The above identified patent application is herein incorporated by reference in its entirety to provide continuity of disclosure.

### BACKGROUND OF THE INVENTION

The present invention relates to chemical compositions. More specifically, the present invention provides a composition for a multi-purpose moisturizer that can be embodied in a cream or in an oil solution.

Many individuals struggle to deal with dry skin and the problems it may cause. For example, eczema, chapped lips, cracked heels, and many other conditions that cause dry skin can be incredibly irritating. Dry scalp skin can lead to thinning hair, which is undesirable for most individuals. Some individuals attempt to relieve dry skin utilizing various topical or ingestible medications. However, such medications are often prohibitively expensive, and many of the medications cause unwanted side effects. There exist moisturizer creams and oils that may be used to alleviate the symptoms of dry skin. However, many of these creams and oils include unwanted chemicals that can cause additional irritation to the affected area. In view of the above, it is necessary and desirable to provide a multi-purpose moisturizer that includes only natural non-harmful ingredients that can be applied to any area of dry skin to reduce irritation and return the skin to a healthy moisture level.

### SUMMARY OF THE INVENTION

In view of the foregoing disadvantages inherent in the known types of moisturizers now present in the prior art, the present invention provides a multi-purpose moisturizer wherein the same can be utilized for providing convenience for the user when alleviating dry skin and returning skin to a healthy moisture level.

One embodiment of the multi-purpose moisturizer is a cream, the composition of which comprises aloe vera butter, African rhea butter, mango butter, cocoa butter, jojoba butter, hemp seed butter, kokum butter, avocado butter, sal butter, sweet almond oil, castor oil, horsetail extract, olive oil vitamin E oil, argan oil, coconut oil, emu oil, rose hip seed oil, ylang ylang essential oil, nettle leaf, sunflower seed oil, jojoba oil, lavender essential oil, apricot oil, grape seed oil, aloe vera gel, chamomile essential oil, and jasmine essential oil.

Another exemplary embodiment of the multi-purpose moisturizer is an oil mixture, the composition of which comprises sweet almond oil, olive oil, coconut oil, avocado oil, jojoba oil, hemp seed oil, vitamin E oil, argan oil, rose hip seed oil, horsetail extract, castor oil, jasmine essential oil, ylang ylang essential oil, nettle leaf, lavender essential oil, emu oil, rhea oil, apricot oil, grape seed oil, sunflower seed oil, aloe vera oil, and chamomile essential oil.

An object of the present invention is to provide a multi-purpose moisturizer that can be applied to any area of dry skin in order to reduce irritation and return the skin to a healthy moisture level.

Another object of the present invention is to provide a multi-purpose moisturizer that can be applied to a dry scalp in order to alleviate dandruff and thinning hair.

Other objects, features, and advantages of the present invention will become apparent from the following detailed description.

### DETAILED DESCRIPTION OF THE INVENTION

Although the characteristic features of this invention will be particularly pointed out in the claims, the invention itself and manner in which it may be made and used may be better understood upon review of the following detailed description.

For the purposes of presenting a brief and clear description of the present invention, the preferred embodiment will be discussed as used for relieving dry skin and combatting hair loss. The following detailed description is intended for representative purposes only and should not be considered limiting in any respect.

The present invention provides a composition for a multi-purpose moisturizer. In one embodiment, the multi-purpose moisturizer has the form of a cream. The cream is composed of various butters mixed with various essential oils. The butter components provide moisturizing properties to the cream, while the essential oils provide moisturizing properties as well as a pleasant scent.

The cream embodiment of the multi-purpose moisturizer composition includes aloe vera butter, mango butter, cocoa butter, jojoba butter, hemp seed butter, kokum butter, avocado butter, sal butter, each of which is present in the composition within a range of 6% to 10% by volume. The cream embodiment also includes African rhea butter and the aloe vera gel, each of which is present in the composition within a range of 10% to 20% by volume. Additionally, the cream embodiment also includes sweet almond oil, olive oil, argan oil, coconut oil, emu oil, sunflower seed oil, jojoba oil, apricot oil, and grape seed oil, each of which is present in the composition within a range of 0.1% and 1.0% by volume. Further, the cream embodiment includes castor oil, horsetail extract, vitamin E oil, rose hip seed oil, ylang ylang essential oil, nettle leaf oil, lavender essential oil, chamomile essential oil, and jasmine essential oil, each of which is present in the composition within a range of 0.1% to 0.5% by volume.

One example of the above-described cream embodiment of the multi-purpose moisturizer consists essentially of 8.16% by volume of aloe vera butter, 16.31% by volume of African shea butter, 8.16% by volume of mango butter, 8.16% by volume of cocoa butter, 8.16% by volume of jojoba butter, 8.16% by volume of hemp seed butter, 8.16% by volume of kokum butter, 8.16% by volume of avocado butter, 8.16% by volume of sal butter, 0.5% by volume of sweet almond oil, 0.17% by volume of castor oil, 0.17% by volume of horsetail extract, 0.5% by volume of olive oil, 0.34% by volume of vitamin E oil, 0.5% by volume of argan oil, 0.5% by volume of coconut oil, 0.5% by volume of emu oil, 0.17% by volume of rose hip seed oil, 0.17% by volume of ylang ylang essential oil, 0.17% by volume of nettle leaf oil, 0.5% by volume of sunflower seed oil, 0.5% by volume of jojoba oil, 0.17% by volume of lavender essential oil, 0.5% by volume of apricot oil, 0.5% by volume of grape seed oil, 12.23% by volume of aloe vera gel, 0.17% by volume of chamomile essential oil, and 0.17% by volume of jasmine essential oil.

In another embodiment of the present invention, the multi-purpose moisturizer has the form of an oil mixture. The oil mixture can be applied to the scalp or other areas where the cream embodiment would be difficult or messy to apply. The combination of oils provide enhanced moisturizing capabilities as well as a pleasant scent.

The oil embodiment of the multi-purpose moisturizer composition includes sweet almond oil, olive oil, coconut oil, avocado oil, jojoba oil, hemp seed oil, argan oil, rose hip seed oil, castor oil, shea oil, apricot oil, grape seed oil, sunflower seed oil, and aloe vera oil, each of which is present in the composition within a range of 5% to 8% by volume. The oil embodiment also includes vitamin E oil, which is present in the composition within a range of 0.5% to 2% by volume. Additionally, the oil embodiment includes jasmine essential oil, ylang ylang essential oil, nettle leaf oil, lavender essential oil, and chamomile oil, each of which is present in the composition within a range of 0.1% to 1% by volume. Further, the oil embodiment includes horsetail extract and emu oil, each of which are present in the composition within a range of 0.5% to 3%.

One example of the above-described oil embodiment of the multi-purpose moisturizer consists essentially of 6.64% by volume of sweet almond oil, 6.64% by volume of olive oil, 6.64% by volume of coconut oil, 6.64% by volume of avocado oil, 6.64% by volume of jojoba oil, 6.64% by volume of hemp seed oil, 1.09% by volume of vitamin E oil, 6.64% by volume of argan oil, 6.64% by volume of rose hip seed oil, 1.64% by volume of horsetail extract, 6.64% by volume of castor oil, 0.55% by volume of jasmine essential oil, 0.55% by volume of ylang ylang essential oil, 0.55% by volume of nettle leaf oil, 0.55% by volume of lavender essential oil, 1.64% by volume of emu oil, 6.64% by volume of rhea oil, 6.64% by volume of apricot oil, 6.64% by volume of grape seed oil, 6.64% by volume of sunflower seed oil, 6.64% by volume of aloe vera oil, and 0.55% by volume of chamomile essential oil.

The multi-purpose moisturizer can be applied to any area of dry skin in order to return the skin to healthy moisture levels. The oil embodiment of the multi-purpose moisturizer may be applied to an area of the skin where hair grows, such as the scalp. In addition to the moisturizing properties, the oil embodiment of the multi-purpose moisturizer may further help to stimulate hair growth when applied to the scalp. Another use of the multi-purpose moisturizer includes applying the oil embodiment to an area of skin, and then applying the cream embodiment shortly thereafter. In this way, the two embodiments of the present invention can be combined to apply maximum relief from irradiation caused by dry skin.

It is therefore submitted that the instant invention has been described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A multi-purpose moisturizer composition embodied in a cream comprising:
aloe vera butter;
African rhea butter;
mango butter;
cocoa butter;
jojoba butter;
hemp seed butter;
kokum butter;
avocado butter;
sal butter;
sweet almond oil;
castor oil;
horsetail extract;
olive oil;
vitamin E oil;
argan oil;
coconut oil;
emu oil;
rose hip seed oil;
ylang ylang essential oil;
nettle leaf oil;
sunflower seed oil;
jojoba oil;
lavender essential oil;
apricot oil;
grape seed oil;
aloe vera gel;
chamomile essential oil; and
jasmine essential oil.

2) The multi-purpose moisturizer composition of claim 1, wherein the amount of each of the aloe vera butter, the mango butter, the cocoa butter, the jojoba butter, the hemp seed butter, the kokum butter, the avocado butter, and the sal butter in the composition is within a range of 6% to 10% by volume.

3) The multi-purpose moisturizer composition of claim 1, wherein the amount of each of the African rhea butter and the aloe vera gel in the composition is within a range of 10% to 20% by volume.

4) The multi-purpose moisturizer composition of claim 1, wherein the amount of each of the sweet almond oil, the olive oil, the argan oil, the coconut oil, the emu oil, the sunflower seed oil, the jojoba oil, the apricot oil, and the grape seed oil in the composition is within the range of 0.1% and 1.0% by volume.

5) The multi-purpose moisturizer composition of claim 1, wherein the amount of each of the castor oil, the horsetail extract, the vitamin E oil, the rose hip seed oil, the ylang ylang essential oil, the nettle leaf oil, the lavender essential oil, the chamomile essential oil, and the jasmine essential oil in the composition is within a range of 0.1% to 0.5% by volume.

6) A multi-purpose moisturizer composition embodied in an oil comprising:
sweet almond oil;
olive oil;
coconut oil;
avocado oil;
jojoba oil;
hemp seed oil;
vitamin E oil;
argan oil;
rose hip seed oil;
horsetail extract;
castor oil;
jasmine essential oil;
ylang ylang essential oil;
nettle leaf oil;
lavender essential oil;
emu oil;
shea oil;
apricot oil;
grape seed oil;
sunflower seed oil;
aloe vera oil; and
chamomile essential oil.

7) The multi-purpose moisturizer of claim 6, wherein the amount of each of the sweet almond oil, the olive oil, the coconut oil, the avocado oil, the jojoba oil, the hemp seed oil, the argan oil, the rose hip seed oil, the castor oil, the shea oil, the apricot oil, the grape seed oil, the sunflower seed oil, and the aloe vera oil in the composition is within a range of 5% to 8% by volume.

8) The multi-purpose moisturizer of claim 6, wherein the amount of the vitamin E oil in the composition is within a range of 0.5% to 2% by volume.

9) The multi-purpose moisturizer of claim 6, wherein the amount of each of the jasmine essential oil, the ylang ylang essential oil, the nettle oil, the lavender essential oil, and the chamomile oil in the composition is within a range of 0.1% to 1% by volume.

10) The multi-purpose moisturizer of claim 6, wherein the amount of each of the horsetail extract and the emu oil in the composition is within a range of 0.5% to 3%.
